(19) United States
(12) Patent Application Publication
(10) Pub. No.: US 2009/0099663 A1
(43) Pub. Date: Apr. 16, 2009

(54) TIBIA PLATFORM IMPLANT

(75) Inventor: Sergio Romagnoli, Milano (IT)

Correspondence Address: ZIMMER TECHNOLOGY - BAKER & DANIELS, 111 EAST WAYNE STREET, SUITE 800, FORT WAYNE, IN, 46802, US

(73) Assignee: ZIMMER GMBH, Winterthur (CH)

(21) Appl. No.: 12/278,104

(22) PCT Filed: Feb. 1, 2007
(86) PCT No.: PCT/EP07/50986

§ 371 Date: Oct. 29, 2008

(30) Foreign Application Priority Data
Feb. 3, 2006 (CH) CH220/06

(51) Int. Cl. A61F 2/38

(52) U.S. Cl. 623/20.3; 623/20.32

(57) ABSTRACT

An implant (6) is provided for implantation in a condyle (21) of a proximal tibia. The implant is shaped in such a way that an anterior edge (63) at least approximately simulates the anterior contour of the tibial plateau (2). The implant is dimensioned such that it does not extend as far as the margin of the tibial plateau in the posterior and lateral directions and is shaped like a plate in such a way that it is free from form-fit anchoring elements and can be pushed in the manner of a drawer into a recess of the tibial plateau and can be cemented. The implant permits maximum preservation of the bone tissue and soft tissue, and the necessary resection and the implantation can both be performed by what is essentially a purely anterior approach.

The invention relates to a tibial implant in accordance with the preamble of claim 1. It furthermore relates to a method for the implanting of the tibial platform implant.

In knee arthroplasty, a complete replacement of the joint is frequently not indicated. Conditions frequently occur, for example as a consequence of defective joint positions or trauma in which the degenerative state is present either only medial or only lateral. In such a case, the use of a monocondylar or unicompartmental knee joint implant can be indicated. The use of a monocondylar implant makes lower losses of bone material necessary, the procedure is less invasive and the cruciate ligaments are not weakened in comparison with a full knee prosthesis.

Due to these advantages, some surgeons also make use of the pair-wise implanting of unicompartmental implants in the treatment of bicompartmental knee effects, particularly when a non-symptomatic patella and/or an intact state of the ligament apparatus is found on a bicompartmental defect of the joint condyles.

A proven unicompartmental knee implant is the ALLEGRETTO by Zimmer. The tibial plateau is resected at one side in the implanting of the tibial component of the ALLEGRETTO. A complete resection of a tibial knee joint condyle takes place in this process. The tibial implant is implanted without an anchorage mechanism, which relieves the bone, on the one hand, and makes proximal access to the tibia superfluous, which in turn contributes to relieving the soft tissues. The ALLEGRETTO tibial component is therefore placed onto the resection surface and can be called an “onlay” or onlay implant. The cortex covering of the ALLEGRETTO tibial component provides a very good stability of the implant. The resection takes place using gages, which ensures the reproducibility of the incision and ultimately also reduces the risk for the patient and for the surgeon. The implant substantially has the shape of a circular section. The implant combines both the articulation surface and a cementing surface provided for the fastening to the bone in one component. In this connection, an embodiment is known in which the cementing side is made of metal and the articulation surface is made of plastic; it is nevertheless a single-part implant, in contrast to implants which generally have a greater height which have a generally metallic base plate which is fastened to the bone, and a meniscus component which is generally made of plastic and which is fastened—generally releasably—to the base plate and provides the articulation surface.

Despite the advantages of conventional unicompartmental knee prostheses, the trend toward ever less invasive surgical procedures and implants which relieve the bone more and more requires a further size reduction of the implants and, associated with this, a further optimization of the surgical procedure. Less invasive accesses reduce the risk of infection and the reconvalescence period, while lower bone losses in primary care leave open more options for the later implanting of revision prostheses.

“Inlay” implants are known, for example, from “The Journal of Bone and Joint Surgery”, Vol. 53 B, No. 2, May 1971. In this process, a pocket is established in the tibial plateau at proximal in a transverse plane surrounded on all sides by bone and an implant is inlaid into it. The cortical bone is thus ideally relieved, on the one hand. On the other hand, the surgeon has to work freehand and without depth limitation in the establishing of the pocket. This makes high demands on the skill of the surgeon and on the view of the resection surface and thus contradicts the minimally invasive approach in a certain manner. Proximal access is likewise difficult since it is hindered by the femoral condyles without incisions which effect the soft tissues. In addition, the implant is only supported by the spongiosa.

U.S. Pat. No. 6,783,550 describes a knee implant in which a tibial component is inserted in the form of a plate into a slit-like cut-out of the tibial plateau open to proximal and anterior with anterior access. The form of the plate is described, for example, as rectangular and otherwise of any desired shape.

A monocondylar tibial implant has become known from WO 00/44316 which includes a tibial plate for fastening to the resection surface of the bone and a meniscus component to be arranged thereon and having an articulation surface.

The implant described in FR 2 686 792 likewise includes a base plate and a meniscus component for articulation. The implant is shaped as a circular section, that is the contours are identical at anterior and posterior.

A tibial implant of the initially named kind is now proposed here which permits a minimally invasive implanting. More specifically, the implant should be set forth such that it can be implanted through a substantially purely anterior access to the proximal tibia. In accordance with a further aspect, the loss of bone tissue should be low. In accordance with another aspect of the invention, the implant should be set forth such that the resected bone volume is filled as completely as possible. This also contributes to spreading the load of the underlying bone as much as possible and so to avoid the danger of the implant sinking into the spongiosa. The implant should in particular also be set forth such that it is supported at least regionally by cortical bone tissue. This means that one endeavors to achieve as many advantageous effects as possible for sacrificed bone tissue. In accordance with yet another aspect, the implant is to be set forth such that the bone incisions required for the implanting are to be established in a reproducible manner guide by gages.

The implant recited in claim 1 has these properties in addition to other advantageous properties.

It is accordingly a question of a plate-like implant having two surfaces which are arranged oppositely disposed and which are provided as articulation surfaces and/or as cementing surfaces. That is, the in particular single-part implant has both a surface for fastening to the resection surface of the tibia and an articulation surface. In this connection, the implant is made completely of plastic; in this respect, the implant can be made, for example, such that both sides are identical and can serve both as an articulation surface and as a cementing surface. In another embodiment, one side is made specifically as a cementing surface and one surface is made specifically as an articulation surface. In another exemplary embodiment, the implant has a plastic surface, in particular a PE surface, which serves as an articulation surface, as well as a metal side fixedly connected to the PE and made, for example, of coarse-blasted titanium, a titanium wire mesh structure such as is marketed, for example, by Zimmer under the name “Sulmesh”, or “Trabecular Metal”, a technology likewise executed by Zimmer for the manufacture of porous metal fabrics in which tantalum is evaporation coated onto a porous carbon structure. In the plan view of one of these two surfaces, the implant has a border which has at least first and second sides which extend substantially in a straight line and which are arranged disposed opposite one another. In this connection, the second side is shorter than the first side. The first side is provided for the purpose of being arranged adjacent to the eminentia, that is toward the center of the tibial plateau, with an anterior-posterior extent, with an implanted implant. The second side is provided to be implanted toward the lateral or medial margin of the tibial plateau. The first side is therefore also called a central side and the second side a peripheral side, with each of the central and peripheral sides being able to come to lie at medial or lateral depending on the condyle onto which the implant is implanted. A third, anterior side of the boundary extends between the two straight sides and has a convex shape. The transitions of the third side to the first and second sides are non-tangential and have an angle different from zero. That is, there are corners there; transition radii having two or three millimeters, for example, or having an even smaller radius, are here also considered corners by the person of ordinary skill in the art since no direct and tangential transition is present from the anterior contour, as the third side, to the first and second sides. The boundary furthermore has a posterior fourth side which, disposed opposite the third side, connects the first and the second sides. The contour of the posterior fourth side is different from the contour of the anterior third side. The anterior third side is provided to come to lie along the anterior-lateral border or the anterior-medial border. The third side therefore in particular has an anatomical contour which is approximated to the anterior-lateral contour and/or to the anterior-medial contour of a plan view of a proximal tibia or which substantially corresponds to the anterior-lateral contour and/or to the anterior-medial contour of a plan view of a proximal tibia. The fourth side of the implant described here is, in contrast, provided to come to lie in a correspondingly machined recess in the tibial plateau and whose shape is therefore not primarily preset by an adaptation to an anatomical geometry, but is rather oriented inter alia with respect to geometries which are easy to establish in the bone and to other practical demands of the surgeon.

The determination of the desired contour which should correspond to the anterior third side or should be approximated to it can be determined, for example, in that a plurality of tibial plateaus of similar size are measured on X-rays or in resections and a mean value is formed. It will naturally be necessary to vary this contour in specific classes and above all in specific size stages in order to be able to cover the total range of the human anatomy in a reasonable approximation.

The mutually oppositely disposed first and second straight sides are also called longitudinal sides in the following.

The first and second sides arranged disposed opposite one another lie substantially parallel to one another in an embodiment of the implant described here; in an alternative embodiment, they are slightly convergent in the anterior-posterior extent, with the angle the two sides enclose within one another preferably lying below 10 degrees and in particular below seven degrees or even below five degrees. In this process, a parallel extent contributes to avoiding double-mating problems in implanting and to being more tolerant toward deviations in the manufacture of the cut-out in the tibial plateau, with the embodiment with anterior-posterior convergent sides also offering advantages in handling for the surgeon depending on the surgical technique chosen; for example, an additional holding effect, a “press-fit effect” can be achieved by hammering such a wedge-shaped implant into a cut-out. Small wedge angles support the friction locking of the implant in the cut-out.

The anatomically contoured third side comprises a convex arcuate segment in one embodiment and a convex circular arcuate segment in a specific embodiment. In an exemplary embodiment, the third side is made as a whole as a segment of an arc. In another embodiment, the third side of the implant comprises a substantially straight-line section which is in particular arranged in addition to an arcuate segment and which in particular lies adjacent to the first side.

In this respect, the invention also includes a set of implants of different sizes which include a straight-line section of the anterior contour in different implant sizes or in which the anterior contour is determined as a whole from the contour of the arc. For example, implants of a smaller size only have the arcuate contour region at anterior, whereas implants of a larger size also have a straight-line contour section within the same implant set.

The anatomically contoured third side of the implant has tangents of which the tangents arranged adjacent to the first and to the second sides are particularly distinguished, with a first tangent arranged adjacent to the first side including a first angle with the first side and a second tangent arranged adjacent to the second side having a second angle with the second side and with the first angle being larger than the second angle. The first angle, for example, lies in a range from 65° to 90°. The angle in particular amounts to at least 75°. In a further embodiment, the angle amounts to a maximum of 85°. In a particular embodiment, the angle is at 82°. The second angle is, for example, less than 45° and, more specifically, a maximum of 30°. The angle, for example, amounts to at least 15° or at least 20° and lies in the range from 20° to 30° in more specific embodiments. The angle the first and second tangents include with one another lies, for example, in the range from 10° to 90° and in more specific embodiments from 30° to 60° or to 70°. The angle the first tangent includes with the first side in particular caused in that the implant is implanted off-center, to the side of the eminentia on the tibial plateau, and should there be matched to the anterior contour of the tibia and thus, with a preset anterior-posterior orientation of the implant, substantially defines the position of the implant on the tibia. The second angle, on the transition to the second side, is also a measure for the width of the implant in comparison with the size of the tibia; the non-tangential transition thus defines that the implant does not reach up to the outer margin of the tibial plateau. The implant is dimensioned such that bone substance, in particular cortical bone substance, is obtained at the—medial or lateral—side margin of the tibial plateau.

In a further embodiment, the contour of the posterior fourth side has at least one straight section which is in particular arranged adjacent to the first side of the boundary, that is adjacent to the side of the boundary provide facing the center of the tibial plateau and is in particular perpendicular thereto.

In a fourth exemplary embodiment, the posterior fourth side includes a convex arcuate contour, in particular a contour of the shape of an arc of a circle, which merges into the second, shorter longitudinal side in a specific embodiment. At the other end of the arcuate contour, it has a tangent which is arranged, for example, perpendicular to the first longer longitudinal side and merges into a straight contour section in a further specific embodiment. The bending radius of the posterior arcuate contour amounts, for example, to at least 5 mm, in particular to at least 8 mm or to at least 10 mm, and is thus substantially different from a rounded or broken corner.

In an embodiment of the implant, at least one of the two surfaces of the implant is rounded concavely or made with a concavely rounded region. This surface is particularly suitable as an articulation surface. In a further development of this embodiment, both surfaces have a concave geometry which is the same in specular symmetry. An implant of this type is thus suitable for medial and lateral implanting on a right and on a left tibia. In this process, the concave design of a surface serves as an articulation surface or as a pocket for the reception of bone cement depending on the implant position.

In other embodiments, the implant has a dedicated articulation surface and a dedicated cementing surface. The cementing surface, for example, has a structure with elevated portions and recesses.

The implant described is in particular free of shape matched anchorage elements such as spigots or webs projecting from the cementing surface and provided for shape matched anchorage in the bone. This feature in particular makes it possible for the implant to be suitable for implanting with a substantially purely anterior access to a correspondingly prepared tibia. In this process, the implant is, for example, introduced into a cut-out worked into a tibial plateau from anterior. The prepared cut-out is bounded laterally and medially in an embodiment by two sagittal planes or by planes which are rotated slightly, at most by up to 20° or 25°, in particular, however, by less than 15° or less than 10°, with respect to a sagittal plane in a transverse plane, i.e. about a longitudinal axis. The posterior bounding of the cut-out lies in front of the posterior bounding of the tibia when viewed in the anterior-posterior direction, that is bone tissue is maintained at posterior. The bounding of the resection depth, that is the distal bounding of the cut-out, is given by a transverse plane. The cut-out is guided through the cortex and is freely accessible at anterior. The implant can then be implanted by a substantially purely anterior-posterior movement into the tibia in the manner of a drawer, in particular due to the freedom from shape matched anchorage elements. The implant is in particular cemented.

To ensure a maximum bone retention, the maximum thickness at the thickest position of an implant between the two surfaces which are used as the articulation and/or cementing surfaces lies at a maximum of 15 mm, in particular below 10 mm. To ensure the strength of the implant, it has a minimal thickness of at least 5 mm at the thinnest position in an embodiment. The depth of the resection to be established corresponds to the maximal thickness of the implant.

The invention will be explained in more detail in the following with reference to embodiments illustrated in the drawing. In this context, the embodiments and the drawing should only be understood in an instructive manner and should not serve for the restriction of the subjects described in the claims. The illustrations in the drawing are simplified; details not necessary for the understanding of the invention have been omitted.

The proximal portion of a tibia 1 is shown in FIG. 1. The tibial joint condyles 21 and 22 as well as the eminentia 23 are arranged on the tibial head 2 with the tibial plateau. For the implanting of an implant of the type set forth above, a segment of the tibial plateaus is removed, starting from the tibial plateau or from a tibial condyle. It is bounded by a lateral and a medial resection surface 31, 32, by a posterior resection surface 33 and by a distal resection surface 34. A shallow cut-out in the tibia results which is open and accessible at proximal and anterior. The required steps can be carried out with the knee in flexion with a substantially purely anterior or anterior-lateral or anterior-medial access. A possibility of establishing the cut-out consists of removing bone material by means of axially operating drilling or cutting tools in a plurality of incision movements made next to one another in a transverse plane with an anterior-posterior feed. In another procedure, the resection surfaces 31 and 32 are established by sagittal incisions or incisions extending approximately sagittally which are guided in a proximal-distal direction, for example, with a suitable tool in the anterior-posterior direction or by longitudinal sections likewise extending in the sagittal plane or in a plane only slightly transversely rotated with respect to the sagittal plane with likewise low proximal space requirements which can be made available with the knee in flexion with high relief of the soft tissue. The resection surface 34 can be established by an incision carried out anterior-posterior in the transverse plane. After removal of the bone section defined in this manner, the recess shown in FIG. 1a arises, for example. The cut-out could equally be established by means of a suitable milling tool, with the feed movements preferably taking place substantially anterior-posterior. The recess shown in FIG. 1 b with a posterior rounding can, for example, be manufactured by means of a guided milling tool, in particular of a finger type, which is guided in a transverse plane and is furthermore guided in a suitable guide track for the posterior boundary. All the incisions are expediently carried out using suitable incision gages. This is very easily possible due to the anterior access of the cut-out to be established and to the reference capability of the incisions at the anterior edge of the tibial plateau.

In the illustrations of FIG. 2, a proximal-distal view of a tibial plateau resected in accordance with the description of FIG. 1b is shown. The anterior and posterior sides of the tibia are marked by A and P respectively. An anterior-posterior axis of the tibia is indicated by 5. 21 and 22 designate the condyles of the tibial plateau. The eminentia is indicated at 23 and the starting points of the cruciate ligaments at 24 and 25. It is absolutely desirable for relieving knee arthoplasty in patients with an intact ligament apparatus to maintain the cruciate ligaments and to weaken the cruciate ligament attachments as little as possible, with in particular a monocondylar prosthetic being best suited to satisfy this demand. The central resection surface 31 facing the eminentia and the peripheral resection surface 32 are made parallel to the anterior-posterior axis in FIG. 2a. The positions of the medial and lateral resection surfaces or the central and peripheral resection surfaces as well as the posterior resection surface with respect to one another can vary in dependence on the geometry of the implant and of the selected surgical technique. For example, the resection surfaces 31 and 32 in no way have to be made in parallel. The surgeon can in particular also round the transitions between the surfaces to avoid notch effects in the tibial plateau. Different possible embodiments contained in the scope of the subjects characterized in the claims, but not restricting them, are shown below in further embodiments. The distal resection surface 34 can be recognized in the plan view. This comprises a spongious region 29 as well as a cortical region 28 to anterior. The cortical bone tissue is maintained at posterior and also peripheral, that is at the side—medial or lateral—margin of the tibial plateau. This ensures a good fixation of an implant to be inserted in the transverse plane.

Peripherally is to be understood here as “facing the side of the tibia” and this can mean medial or lateral depending on the position of the implant: if the implant is implanted on a medial condyle, the medial side of the tibia is to be understood by this. Vice versa, if the implant is implanted at the lateral side of the tibia, peripheral is to mean the same as lateral. Analogously, central is to be understood as the orientation which faces the eminentia in a transverse plane.

FIGS. 2b and 2c show embodiments of the invention in which the resection for the implanting of the implant has been prepared rotated by an angle 51 in the transverse plane.

FIG. 3 illustrates the resection of a tibial plateau in accordance with FIG. 1a.

A plan view of an exemplary implant is shown in FIG. 4 which is suitable for the implantation into a tibia prepared in accordance with FIGS. 1b and 2. The implant 6 is shown in the plan view of a first surface 7. The boundary of the implant in this plan view comprises a first side 61 and a second side 62 which are provided for implanting at the resection surfaces 31 and 32. In accordance with the definition set forth above, 61 is the central side of the implant which is provided for implanting adjacent to the eminentia and 62 is the peripheral side. The third side 63 of the implant has a convex arcuate extent, with the local radius of the arc being marked by R. The third, anterior side 63 of the implant is anatomically shaped as much as possible and is approximated as closely as possible to the contour of a proximal tibia or tibial platform respectively in the anterior-peripheral region—that is anterior-lateral and/or anterior-medial region—that is in the region in which the resection passes through the cortical bone at anterior. In special embodiments, the arc is an arc of a circle or an arc of an ellipse. In another embodiment, the anterior contour of the implant comprises a plurality of arcs of a circle of different radii and/or arcuate sections and sections with a substantially straight extent. The third side has two characteristic tangents, namely a first tangent 66 adjacent to the first side 61 and a second tangent 67 adjacent to the second side 62. They enclose the angles 611 and 612 with the first or second sides respectively and the angle 613 with one another. In the embodiment shown, the sides 61 and 62 are parallel, for example, and the angle 611 lies in the range between 80° and 90°, whereas the angle 612 lies approximately at 30°. Ranges of the angles are set forth in the claims. It is self-explanatory for the skilled person that the angle 612 which includes the tangent 67 of the anterior contour with the second side 62 also has a dependence on the width of the implant in relation to the width of the total tibial plateau. The implant proposed here is in particular actually characterized in that the width is always small enough still to maintain bone substance at the lateral margin of the tibial plateau. The anterior third side therefore in particular does not merge tangentially into the second side; the angle 612 in an implant of the type suggested here always amounts, for example, to at least 10° or 15°. When the transitions from the anterior side 63 to the lateral and/or medial side 61 and 62 are rounded, the corner tangents 66 and 67 can be determined by extrapolation of the anterior contour. The posterior side 64 of the implant includes a straight section 641 as well as an arcuate section 642 which, in this example, is an arc of a circle having the radius r and has tangential transitions both to the second side 62 and to the straight section 641. The arcuate contour section inter alia gives the surgeon the possibility of still rotating the implant a little in the resected recess. The straight contour sections adjoining the central first side 61 at posterior at least approximately at a right angle facilitates the machining of the bone and increases the support surface of the implant. The surface 7 is, for example, an articulation surface.

FIG. 5 shows example incisions along a line V-V through the implant of FIG. 4. In a first embodiment in accordance with FIG. 5a, both surfaces 7 and 8 have identical contours in specular symmetry. Both the surface 7 and the surface 8 can thus be implanted as an articulation surface. the respective oppositely disposed concave pocket then serves for the reception of bone cement on the implanting.

An implant formed in this manner is suitable for both medial and lateral implanting in a right or left tibia. In a deviation from this, the implant shown in FIG. 5b has an articulation surface 7 with a concave region and a substantially planar cementing surface 8. In this example, the implant has a polyethylene articulation element 68 and a layer 69 at the fastening side which is made, for example, from titanium, a porous metal “foam” such as “Trabecular Metal” or a titanium wire mesh such as is marketed by Zimmer under the name “Sulmesh”. In this respect, the plastic and metal are, however, firmly connected to one another; it is a single-part implant. The cementing surface has a structure with elevated portions and recesses 81 in an embodiment of the implant in accordance with FIG. 5c to achieve an improved association with the bone cement. The implant shown in FIG. 5c and also that of FIG. 5a consists of a single material, for example of a polyethylene material, in particular of highly cross-linked polyethylenes and furthermore polyethylenes stabilized with a-tocopherol; the geometry shown in FIG. 5b can naturally also be completely manufactured from one material, for example, from one of the named polyethylene materials. In this respect, the different embodiments shown in a section in a sagittal plane in FIG. 5 also show examples for different embodiments of the articulation surfaces. The articulation surfaces are spherically concavely shaped by way of example in FIGS. 5a and 5b, that is the articulation surface is also shown to be contoured in a section in a frontal plane. The articulation surface is shaped cylindrically concavely in FIG. 5c.

It must generally be stated that the implanting of the implant described and claimed here takes place in cemented form. It must also be noted that in an embodiment the implant is free from anchorage elements which are provided for a shape matching connection to the bone prepared for implanting. It is thus possible to carry out the resection of the tibial platform with a substantially purely anterior access and to insert the implant into the cut-out of the tibia in the manner of a drawer with an anterior access. Since the total access substantially takes place substantially at anterior or anterior-lateral or anterior-medial and no proximal access to the tibia is required, a maximum relief of the ligament apparatus and of the soft tissue apparatus also results. With the knee in flexion, the resection of the femur and the implanting of a monocondylar femoral component can also still take place by the same anterior access. This again emphasizes the minimally invasive approach of the implant and of the surgical procedure.

FIG. 6 shows by way of example two embodiments of implants 6 respectively seen from the articulation side and from the cementing side. 7 designates the articulation surface; 8 designates the cementing surface. The recesses 81 for the reception of the bone cement have different configurations.

FIG. 7 shows an example of a tibia 1 with an implanted implant 6. As can be recognized, the anterior side 63 of the implant 6 replicates the anterior edge of the tibial plateau in a good manner. In the anterior region, the implant comes to lie on the cortical portion of the distal resection surface, as can be recognized in the joint view with FIGS. 2 and 3. The implant hereby has good anterior support. The surgeon can also improve the matching of the anterior implant contour to the anterior contour of the tibia with a predetermined medial-lateral position of the implant by a slight rotation of the implant and optionally by means of a sloping position of the resection in accordance with FIGS. 2b and 2c or 3b and 3c.

FIG. 8 shows some exemplary dimensional definitions for the description of a generally shaped implant of the described type: central length L1; anterior width bA, posterior width bP; anterior contour height H. The anterior contour is comprised in the example shown of a straight section 631 and an arcuate section. The arcuate section has a generally position-dependent radius R(φ); if the radius is independent of the position, it is an arc of a circle. The transitions between all sides are rounded. The end tangents 66 and 67 are determined by extrapolation of the sides up to a virtual point of intersection at this point of intersection. A rounded posterior contour section having the radius r is drawn in a dashed line and merges tangentially into the second side 62 and into a straight posterior contour section.

Specific embodiments have the following dimension parameters to achieve a particularly good replication of the anatomical contour of the anterior edge of the tibial platform by the implant:

The anterior width of the implant lies in the range from 16 mm to 37 mm. and in particular 34 mm, and in particular in the range from 18 mm to 33 mm, and varies, for example, within an implant family in dependence on the implant size. In an embodiment, the curved anterior contour region has an arcuate shape and the radius of the anterior arc of the circle is in the range from 17 mm to 40 mm, and in particular from 24 to 33 mm. The ratio of anterior width to the radius of the arc of a circle lies, for example, in the range from 0.8 to 1.2, and in more specific embodiments in the range from 0.8 to 1. In a specific embodiment, the ratio of the anterior radius of the arc of a circle to the anterior width is from 1 to 1.3. The contour height lies in the range from 3.3 mm to 37 mm. and in particular 10 mm to 25 mm and furthermore 13 mm to 20 mm. The ratio of the contour height to the anterior width ranges, for example from 0.55 to 0.75 and in particular from 0.60 to 0.70. The posterior radius is, for example, in the range from 10 mm to 30 mm and in particular in the range from 12 mm to 25 mm and furthermore 14 mm to 22 mm. The posterior radius ranges in the ratio to the anterior width from 0.60 to 0.85 and in particular from 0.65 to 0.78. The implant length ranges from 30 mm to 60 mm and in particular from 35 mm to 57 mm or 55 mm. The ratio of implant length to anterior width is in the range from around 1.7 to 1.9.

In an embodiment, the anatomical side 63 of the implant is formed by an arc of a circle and by a segment 631 extending in a straight line and arranged adjacent to the first side. The length of this segment extending straight lies at around 4 mm to 10 mm. The end tangent of the third side at the first side in particular includes an angle with this in the range from 65° to 90°; this angle is in particular in the range from 75° or 80° to 85° and it in particular amounts to 82°. The end tangent at the second side in particular includes an angle with it in the range from 10° to around 35°.

An exemplary set of implants includes implants of different sizes, with the anterior width being in the range from 16 mm, and in particular from 19 mm, to 33 mm, for example. The ratio of an anterior radius of an arc of a circle to the anterior width varies in the range from 1 to 1.3°, with this ratio reducing in a specific embodiment from the smallest implant size to the largest implant size. The ratio of contour height to anterior width is in the range from 0.6 to 0.7. The ratio of posterior radius to the anterior width is in the range from 0.65 to 0.8 and the ratio from the length to the anterior width is in the range from 1.65 to 1.9. With all these ratios, the largest values occur with the smallest implant sizes and the smallest values occur with the largest implant sizes. In an embodiment, the anterior contour is continuously curved in the smallest implant sizes, whereas the larger implant sizes also have a straight anterior contour region. The angle a tangent of the anterior contour at the central first side includes with the central first side is in the range from 80° to 85°, in particular 82°, and is furthermore in particular constant within the implant set for all implant sizes.

The implant is made in an embodiment from a polyethylene, in particular from a highly cross-linked polyethylene and/or an ultra-highly molecular polyethylene. In a further development, the polyethylene is doped with a-tocopherol (vitamin E). In an embodiment, the thickness of the implant between the two surfaces then does not lie below 5 mm at any point. In a further development of this embodiment, the thickness of the implant does not lie above 15 mm at any point and the maximum thickness is, for example, in a range up to a maximum of 13 mm, to a maximum of 10 mm, or to a maximum of 8 mm.

In the light of the statements made here, further embodiments of the implant characterized in the claims which cannot be shown here in an exclusive manner become known to the skilled person.

### REFERENCE NUMERAL LIST

1 tibia
2 tibial head
5 anterior-posterior axis
6 implant
7 first surface; articulation surface
8 second surface, cementing surface
21 joint condyle
22 joint condyle
23 eminentia
24 attachment point of the anterior cruciate ligament
25 attachment point of the posterior cruciate ligament
28 cortex
29 spongiosa
31 central side (medial or lateral) resection surface
32 peripheral side (medial or lateral) resection surface
33 posterior resection surface
34 distal resection surface
51 angle of the implant axis to the anterior-posterior axis
61 first, central (medial or lateral) side of the implant
62 second, peripheral (medial or lateral) side of the implant
63 anterior side of the implant
64 posterior side of the implant
66 first (central) tangent of the anterior contour
67 second (peripheral) tangent of the anterior contour
81 recess for the reception of bone cement
611 angle between the first tangent and the first side
612 angle between the second tangent and the second side
613 angle between the first tangent and the second tangent
631 anterior straight contour region
641 posterior straight contour region
642 posterior curved contour region
A anterior
P posterior
R anterior arc radius
r posterior arc radius
L1 central length
bA anterior width
bP posterior width

1-21. (canceled)

22. A one-part tibial platform inlay implant, comprising two surfaces which are arranged oppositely disposed and of which at least one is provided at least as an articulation surface and at least one is provided at least as a cementing surface, and said implant having a boundary in the plan view of one of the two surfaces which has at least one first side and a second side extending substantially straight and disposed opposite one another, and wherein the boundary comprises a convex arcuate contour at an anterior third side and has a transition to the first side and to the second side which is non-tangential and has a transition angle different from zero, a posterior fourth side of the boundary having a contour different from the third side.

23. A tibial platform implant in accordance with claim 22, wherein the first side is longer than the second side.

24. A tibial platform implant in accordance with claim 22, wherein the contour of the boundary at the third side is approximated to the anterior-lateral contour and/or to the anterior-medial contour of a plan view of a proximal tibia and in particular substantially corresponds to it.

25. A tibial platform implant in accordance with claim 22, wherein the contour of the anterior third side includes an arc of a circle and is in particular in the shape of an arc of a circle as a whole.

26. A tibial platform implant in accordance with claim 22, wherein the third side comprises a section which extends substantially in a straight line and which is in particular arranged adjacent to the first side and at which a convexly curved contour section in particular adjoins tangentially.

27. A tibial platform implant in accordance with claim 22, wherein the first side and the second side are parallel to one another.

28. A tibial platform implant in accordance with claim 22, wherein the posterior fourth side comprises a straight section which is in particular arranged adjacent to the first side and is in particular arranged perpendicular thereto.

29. A tibial platform implant in accordance with claim 22, wherein the posterior fourth side comprises a convex arcuate contour which is in particular arcuate.

30. A tibial platform implant in accordance with claim 22, wherein at least one surface is concave.

31. A tibial platform implant in accordance with claim 22, wherein both surfaces have the same concave geometry of specular symmetry.

32. A tibial platform in accordance with claim 22, wherein one of the surfaces is made as a dedicated articulation surface and the other surface is made as a dedicated cementing surface.

33. A tibial platform implant in accordance with claim 32, wherein the cementing surface has a structure with elevated portions and recesses.

34. A tibial platform implant in accordance with claim 22, wherein the implant is free of anchorage elements, in particular of shape matched anchorage elements.

35. A tibial platform in accordance with claim 22, wherein it is suitable for implantation into a correspondingly prepared tibia with a substantially purely anterior access.

36. A tibial platform implant in accordance with claim 22, wherein a thickness measure of the implant measured between the first and the second surfaces amounts to a maximum of 10 mm.
